(19) United States
(12) Patent Application Publication
(10) Pub. No.: US 2009/0299345 A1
(43) Pub. Date: Dec. 3, 2009

(54) SYSTEM AND METHOD FOR RESHAPING A CORNEA USING A COMBINATION OF LIOB AND STRUCTURAL CHANGE PROCEDURES

(76) Inventors: Josef F. Bille, Heidelberg (DE); Luis Antonio Ruiz, Bogota (CO)

Correspondence Address: NYDEGGER & ASSOCIATES, 348 OLIVE STREET, SAN DIEGO, CA, 92103, US

(21) Appl. No.: 12/127,539

(22) Filed: May 27, 2008

(51) Int. Cl. A61B 18/18

(52) U.S. Cl. 606/5

(57) ABSTRACT

A system and method for reshaping and altering the cornea of an eye employs a laser beam for two different purposes. For one, the laser beam is configured to perform Laser Induced Optical Breakdown (LIOB) on selected tissue in the stroma of the cornea. This generally weakens the stromal tissue and allows intraocular pressure and bio-mechanical forces in the eye to reshape the cornea. For the other, the laser beam is configured to cause Permanent Structural Change (PSC) on selected tissue in the stroma of the cornea. This alters the tissue density of the selected PSC stromal tissue to change its refractive index. In combination, the LIOB and PSC provide for corrected vision.

### FIELD OF THE INVENTION

The present invention pertains generally to systems and methods for reshaping and structurally altering the cornea of an eye to improve a patient's vision. More particularly, the present invention pertains to systems and methods that employ a laser beam for different purposes in an ophthalmic surgical procedure. The present invention is particularly, but not exclusively, useful as a system and method for reshaping the cornea of an eye, wherein Laser Induced Optical Breakdown (LIOB) of selected LIOB stromal tissue is accomplished to reshape the eye, and wherein Permanent Structural Change (PSC) of selected PSC stromal tissue is accomplished to alter the cornea by changing the refractive index of the tissue selected for PSC.

### BACKGROUND OF THE INVENTION

As laser system capabilities have become better understood, the use of lasers to change the physical characteristics of a resilient transparent material, such as the cornea of an eye, has become a more acceptable surgical procedure for vision correction. For purposes of discussion, refractive surgical procedures that are particularly useful for ophthalmic surgery can generally be grouped into either of two categories. The procedures most commonly used at the present time are those that result in a reshaping of the cornea. In these procedures, tissue is selectively removed from the cornea. Consequently, the cornea changes its shape to achieve changes in the refractive properties of the cornea. Procedures in the other category, however, do not rely on tissue removal. Instead, they rely on structurally changing the corneal tissue to alter the refractive properties of the tissue. Though both type procedures can result in a predictable vision correction, they are quite different from each other. And, importantly, they require different laser beam energy configuration (i.e. volumetric power densities).

Reshaping a cornea by removing tissue from the cornea is typically accomplished by performing Laser Induced Optical Breakdown (LIOB) of tissue. Simply stated, LIOB results in the photoablation (i.e. destruction) of tissue. The consequence of this is that tissue is effectively removed from the cornea. Thus, the cornea is somehow weakened. Internal forces inside the eye (i.e. intraocular pressure, and biomechanical forces) will then reshape the cornea to achieve the desired optical effect. To achieve LIOB, however, the energy in the laser beam must be above the LIOB threshold of the tissue. Typically, the required energy level for LIOB will be above about 500 nJ. As implied above, LIOB is associated with procedures in the first (i.e. cornea reshaping) category.

For the second category of surgical procedures (i.e. tissue altering), corneal tissue is not removed, and the cornea is not reshaped. Instead, structural aspects of corneal tissue are physically altered. This alteration consequently changes the refractive index of the tissue. In comparison with LIOB, this requires much less energy in the laser beam (e.g. 25 nJ vis-à-vis 500 nJ). Importantly, in order to effectively alter the structure of tissue in the cornea, the energy level must be above a threshold level where the tissue is responsive to the laser beam. In this case, however, rather than photoablating tissue, as happens with LIOB, tissue is merely altered (i.e. melted into a semi-liquefied state) by heat from the laser beam. In this heating process, the visco-elastic properties of the tissue are altered. Specifically, the tissue's inter-molecular bonds are changed to create a structure having denser tissue, with a higher refractive index. This phenomenon was first recognized in U.S. Pat. No. 4,907,586 which issued to Bille et al. for an invention entitled “Method for Reshaping the Eye.” Hereinafter, in the context of the present invention, a surgical procedure wherein tissue is altered to change its refractive index is referred to as a Permanent Structural Change (PSC) procedure.

In light of the above, it is an object of the present invention to provide a system and method for reshaping and altering the cornea of an eye that employs LIOB and PSC as complementary surgical procedures for the correction of vision defects. Another object of the present invention is to provide a system and method for using PSC to enhance the surgical outcome obtained by LIOB. Still another object of the present invention is to provide a system and method for reshaping and altering the cornea of an eye that is easy to use, is simple to implement and is comparatively cost effective.

### SUMMARY OF THE INVENTION

In accordance with the present invention, a system and method are provided for reshaping and altering the cornea of an eye to correct vision defects. More specifically, for the present invention laser beams having different energies are used for two different purposes. Essentially, these purposes differ from each other in the effect they have on stromal tissue in the cornea.

In a first mode of operation, a laser unit generates a first laser beam for the purpose of performing Laser Induced Optical Breakdown (LIOB) on selected tissue in the stroma. In a second mode of operation, the laser unit generates a second laser beam (e.g. reconfigures the first laser beam) for the purpose of causing a Permanent Structural Change (PSC) in selected tissue of the stroma. As envisioned for the present invention, LIOB is performed on stromal tissue to generally weaken selected portions of the stroma. This weakening of the tissue will then allow intraocular pressure and bio-mechanical forces in the eye to reshape the cornea. On the other hand, PSC is performed on stromal tissue with the intention of altering the selected tissue in a manner that will change its refractive index. Together, these procedures can be complementarily employed to correct vision defects.

For purposes of the present invention, the first laser beam and the second laser beam will each have pulses of approximately 500 fs duration, and each will have a pulse repetition rate of approximately 80 kHz. Further, the first laser beam and the second laser beam are each generated with a numerical aperture (N. A.) of 0.2, corresponding to a focal point spot size of approximately four microns. The primary difference between the laser beams will be that they operate at different energy levels. More specifically, the first laser beam is envisioned to operate at an energy level that is above the LIOB threshold of the selected LIOB tissue. Preferably, energy in the first laser beam will be greater than approximately 500 nJ. On the other hand, the second laser beam will operate at an energy level that is below the LIOB threshold, but above the Permanent Structural Change threshold of the selected PSC tissue. Preferably the energy in the second laser beam will be approximately 25 nJ. As envisioned by the present invention, the second laser beam can be configured from the first laser beam by using a beam splitter that will split the first laser beam into a plurality of different beams.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of a laser system positioned relative to the cornea of an eye for performance of LIOB and PSC surgical procedures in accordance with the present invention;

FIG. 2A is a cross section view of a cornea of an eye showing vertical cuts in the cornea caused by LIOB;

FIG. 2B shows a reshaping of the cornea as a result of cuts introduced by LIOB in FIG. 2A; and

FIG. 2C shows areas/volumes of tissue altered by PSC to enhance the surgical outcome of LIOB reshaping, such as is shown in FIG. 2B.

### DESCRIPTION OF THE PREFERRED EMBODIMENTS

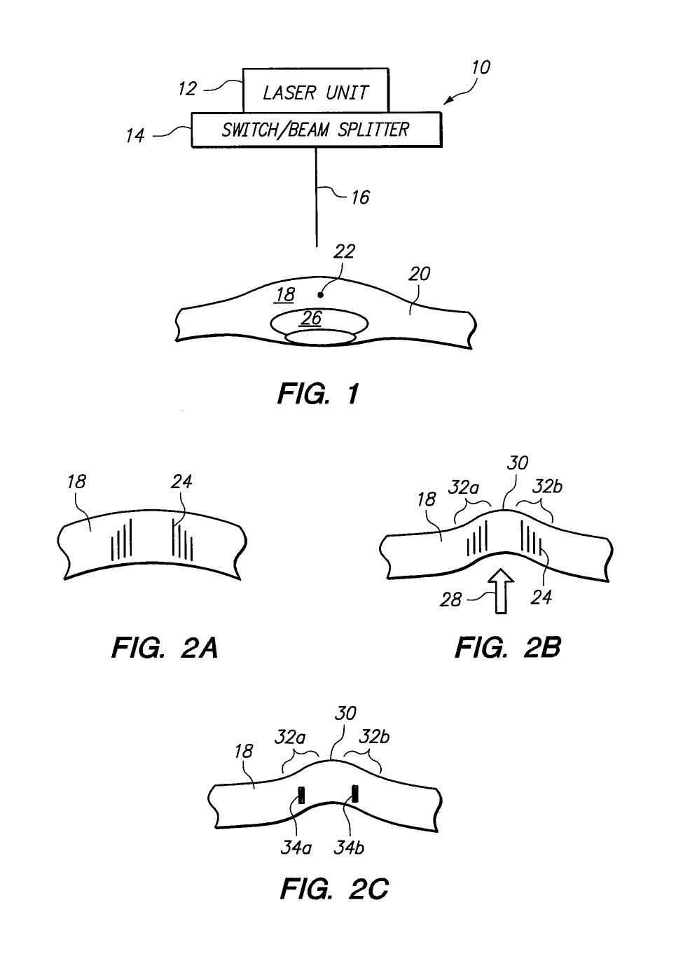

Referring initially to FIG. 1, a system in accordance with the present invention is shown and is generally designated 10. In FIG. 1 it will be seen that the system 10 basically includes a laser unit 12 with a switch 14. For purposes of the present invention, the laser unit 12 is preferably of a type that is capable of generating a laser beam 16 with femto-second pulses. Preferably, each pulse in the beam 16 will have a duration of approximately 500 fs with a pulse repetition rate of approximately 80 kHz. Further, the laser unit 12 will provide a numerical aperture (N. A.) of around 0.2 for a corresponding spot size of approximately four microns.

As envisioned for the present invention, the laser unit 12 needs to be capable of generating a laser beam 16 having either an energy level (i.e. volumetric power density) of either around 500 nJ or around 25 nJ. This change in energy level can be accomplished in either of several ways. For one, the switch 14 can change operation of the laser unit 12 from one laser source to another. For another, the laser beam 16 having around 500 nJ of energy can be divided by a beam splitter (designated 14 with the switch) that will provide for a plurality of individual beams having respectively less energy.

Insofar as the different energy levels of the beam 16 are concerned, it is important that the laser unit 12 of system 10 be able to operate in a first mode with a beam energy that is above the Laser Induced Optical Breakdown (LIOB) threshold of tissue in the cornea 18 of an eye 20. As indicated above, this energy level is at, or above, approximately 500 nJ. On the other hand, the laser unit 12 must also be able to operate in a second mode where the beam energy is above the Permanent Structural Change (PSC) threshold for tissue in the cornea 18. As indicated above, this energy level is generally greater than, but preferably near, about 25 nJ.

For the present invention, it is envisioned that LIOB can be performed on selected tissue in the cornea 18 with a resultant refractive change of approximately 2 diopters. The present invention also envisions that PSC can be performed on selected tissue in the cornea 18 with a local resultant refractive change of approximately 0.5 diopters. In general, this will be so regardless wherein the focal point 22 of the laser beam 16 is located in the cornea 18. While it is recognized that PSC may be performed on a layer across the entire cornea 18, it may well be preferable to employ PSC more selectively. In any event, the present invention envisions the complementary employment of both LIOB and PSC.

An exemplary sequence for the employment of an LIOB/PSC procedure in accordance with the present invention is shown in FIGS. 2A, 2B and 2C. Beginning with FIG. 2A, it is indicated that a plurality of cuts 24 can be made into the tissue of cornea 18, using LIOB techniques for the purpose of weakening the cornea 18. Preferably, this LIOB portion of the procedure is conducted in a manner disclosed in U.S. patent application Ser. No. 11/958,202, for an invention entitled “Method for Intrastromal Refractive Surgery”, which was filed on Dec. 17, 2007, and which is assigned to the same assignee as the present invention. As is indicated in FIG. 2B, once the cuts 24 have been made in the cornea 18 by LIOB, the cornea 18 is weakened. Intraocular pressure from the anterior chamber 26 of the eye 20 (see FIG. 1) will then act against the weakened cornea 18 as indicated by the arrow 28, to reshape the cornea 18. In the example given, the anterior surface 30 of cornea 18 has experienced a bulge with consequent depressions 32a, 32b. It can happen, that these depressions 32a, 32b may not complete the required refractive corrections. If so, the present invention envisions employing a PSC procedure on selected tissue in the areas/volumes 34a, 34b to enhance the LIOB procedure and further improve the outcome for refractive surgery.

While the particular System and Method for Reshaping a Cornea Using a Combination of LIOB and Structural Change Procedures as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A system for reshaping and altering a cornea which comprises:
a laser unit for generating a laser beam, and for directing the laser beam to a focal point in the stromal tissue of a cornea; and
a switch mounted on the laser unit for changing operation of the laser unit between a first mode wherein the laser beam is configured to perform Laser Induced Optical Breakdown (LIOB) on stromal tissue selected for LIOB, and a second mode wherein the laser beam is configured to cause a Permanent Structural Change (PSC) in stromal tissue selected for PSC, wherein LIOB results in a cutting of the LIOB stromal tissue and PSC results in a change in refractive index of the PSC stromal tissue to enhance the LIOB results.

2. A system as recited in claim 1 wherein the laser beam has a first energy level in the first mode of operation that weakens stromal tissue in the cornea to allow intraocular pressure and bio-mechanical forces in the eye to reshape the cornea.

3. A system as recited in claim 2 wherein the laser beam has a second energy level in the second mode of operation that alters the tissue density of the selected PSC stromal tissue to change its refractive index.

4. A system as recited in claim 1 wherein the first mode is characterized by a laser beam configuration for operation at a first energy level above the LIOB threshold of the selected tissue with energy greater than approximately 500 nJ.

5. A system as recited in claim 4 wherein the laser beam configuration includes pulses of approximately 500 fs duration having a pulse repetition rate of approximately 80 kHz.

6. A system as recited in claim 1 wherein the second mode is characterized by a laser beam configuration for operation at a second energy level below the LIOB threshold and above the Permanent Structural Change threshold of the selected tissue with energy of approximately 25 nJ.

7. A system as recited in claim 1 wherein the laser unit operates with a numerical aperture (N. A.) of 0.2, corresponding to a focal point spot size of approximately four microns.

8. A system for reshaping and altering the cornea of an eye which comprises:
a means for generating a first laser beam to perform Laser Induced Optical Breakdown (LIOB) on tissue selected for LIOB in the stroma of the cornea, wherein LIOB results in a cutting of the LIOB stromal tissue;
a means for generating a second laser beam to cause Permanent Structural Change (PSC) on tissue selected for PSC in the stroma of the cornea, wherein PSC results in a change in refractive index of the PSC stromal tissue to enhance the LIOB results; and
a means for changing between the first laser beam and the second laser beam to selected focal points for respective LIOB and PSC in the stroma of the cornea.

9. A system as recited in claim 8 wherein the means for generating a first laser beam to perform LIOB has a first energy level that weakens stromal tissue in the cornea to allow intraocular pressure and bio-mechanical forces in the eye to reshape the cornea.

10. A system as recited in claim 9 wherein the means for generating a second laser beam to cause PSC has a second energy level that alters the tissue density of the selected PSC stromal tissue to change its refractive index.

11. A system as recited in claim 8 wherein the first laser beam and the second laser beam include pulses of approximately 500 fs duration having a pulse repetition rate of approximately 80 kHz.

12. A system as recited in claim 11 wherein the first laser beam operates at a first energy level above the LIOB threshold of the selected LIOB tissue with energy greater than approximately 500 nJ, and the second laser beam operates at a second energy level below the LIOB threshold and above the Permanent Structural Change threshold of the selected PSC tissue with energy of approximately 25 nJ.

13. A system as recited in claim 8 wherein the first laser beam and the second laser beam are generated with a numerical aperture (N. A.) of 0.2, corresponding to a focal point spot size of approximately four microns.

14. A system as recited in claim 8 wherein the means for generating the second laser beam includes a beam splitter.

15. A method for reshaping and altering the cornea of an eye which comprises the steps of:
generating a first laser beam to perform Laser Induced Optical Breakdown (LIOB) on tissue selected for LIOB in the stroma of the cornea, wherein LIOB results in a cutting of the LIOB stromal tissue;
generating a second laser beam, in sequence with generation of the first laser beam, to cause Permanent Structural Change (PSC) on tissue selected for PSC in the stroma of the cornea, wherein PSC results in a change in refractive index of the PSC to enhance the LIOB results; and
changing between the first laser beam and the second laser beam to respectively selected focal points in the stroma of the cornea.

16. A method as recited in claim 15 wherein the first laser beam has a first energy level to perform LIOB that weakens stromal tissue in the cornea to allow intraocular pressure and bio-mechanical forces in the eye to reshape the cornea.

17. A method as recited in claim 16 wherein the second laser beam has a second energy level to perform PSC that alters the tissue density of the selected PSC stromal tissue to change its refractive index.

18. A method as recited in claim 15 wherein the first laser beam and the second laser beam include pulses of approximately 500 fs duration having a pulse repetition rate of approximately 80 kHz.

19. A method as recited in claim 18 wherein the first laser beam operates at a first energy level above the LIOB threshold of the selected LIOB tissue with energy greater than approximately 500 nJ, and the second laser beam operates at a second energy level below the LIOB threshold and above the Permanent Structural Change threshold of the selected PSC tissue with energy of approximately 25 nJ, and further wherein the first laser beam and the second laser beam are generated with a numerical aperture (N. A.) of 0.2, corresponding to a focal point spot size of approximately four microns.

20. A method as recited in claim 15 wherein the step of generating the second laser beam is accomplished using a beam splitter.
